(19) United States
(12) Patent Application Publication
(10) Pub. No.: US 2017/0095312 A1
(43) Pub. Date: Apr. 6, 2017

(54) MARKERS INCLUDING MAGNETIC TRANSPONDERS WITH INCREASED RADIOGRAPHIC VISIBILITY

(71) Applicant: Varian Medical Systems, Inc., Palo Alto, CA (US)

(72) Inventors: Dirk Tenne, Bellevue, WA (US); Martin Vogel, Seattle, WA (US)

(21) Appl. No.: 14/874,273

(22) Filed: Oct. 2, 2015

(51) Int. Cl. A61B 90/00; A61L 31/18; A61L 31/02

(52) U.S. Cl. CPC A61B 90/39; A61L 31/022; A61L 31/18; A61B 2090/3958; A61B 2562/162; A61B 2090/3966; A61B 2090/3762; A61B 2090/3987

(57) ABSTRACT

A wireless marker including a magnetic transponder employs a high-Z material to improve the visibility of the marker in radiographic images and the electrical performance of the marker for tracking with a tracking system. The magnetic transponder includes a ferromagnetic core and a coil of a conductive wire comprising the high-Z material around the ferromagnetic core. The magnetic transponder generates a wirelessly transmitted magnetic field in response to wirelessly transmitted excitation energy.

### TECHNICAL FIELD

Embodiments of this disclosure relate generally to accurately locating and tracking a target in a body to which radiation is delivered and/or other medical procedures are performed. In particular, various embodiments of wireless markers including a magnetic transponder with increased radiographic visibility and improved electrical performance are described.

### BACKGROUND

Radiation therapy and many other medical procedures require locating a target with a high degree of precision to limit collateral damage to healthy tissue around the target. Precisely locating a target is particularly important in radiotherapy because it is desirable to accurately determine the accumulated dosage applied to the target and detrimental to expose adjacent healthy body parts to radiation. In radiotherapy of prostate cancer, for example, it is detrimental to irradiate the colon, bladder, or other neighboring body parts with high-intensity radiation. Surgical applications, such as breast surgery and other procedures involving soft tissue, also require knowing the precise location of a target because a lesion in soft tissue is not necessarily fixed relative to external landmarks on the patient.

Fiducial markers have been used to locate targets in a patient. Fiducial markers are solid, inert metals that can be implanted in a patient at or near a target. One example is VISICOIL™ gold markers. The positions of fiducial markers implanted in a patient can be determined periodically using x-ray imaging systems. While fiducial markers are useful in localizing targets within a patient, they are passive markers and do not provide active real-time location information during radiotherapy or other medical procedures.

Active markers that generate detectable signals have been used to locate selected targets in real time. Many active markers are implantable in a patient and hard-wired to a power source or other equipment external to the patient. These hard-wired markers are removed from the patient after a procedure or a series of procedures are concluded. The removal process, however, requires an additional invasive procedure to the patient's body. Wireless active markers have been developed to be implanted in a patient at or near a selected target. Wireless active markers are typically activated or energized by an energy source external to a patient. In response to the excitation energy, the active markers generate a detectable signal that is wirelessly transmitted to a sensing system outside the patient. Some wireless markers contain a power source, such as a battery, that provides the power to generate a signal detectable from outside the patient's body. Some wireless markers contain a magnetic transponder to generate a resonating magnetic field detectable from outside the patient's body.

One challenge of using wireless active markers with resonating magnetic circuits is determining the relative location between the markers and the target so that the target can be tracked during radiotherapy or other medical procedures. Accurately determining the location of markers relative to the target is a precondition for accurately tracking the target based on the resonating magnetic field generated by the markers. One reason that it is difficult to accurately determine the location of markers relative to the target is that it can be difficult to identify magnetic resonating markers in radiographic images. The markers are difficult to see in radiographic images because they should be very small so that they may be implanted for an extended period of time, and/or, they may not be sufficiently visible in high voltage radiation applications such as megavolt radiation imaging. Moreover, even when a magnetic marker can be identified in an image, it can still be challenging to determine the orientation of the magnetic field generated by the markers relative to the target because it is often difficult to determine the orientation of the markers in the image. Therefore, conventional implantable markers with resonating magnetic circuits may be difficult to use in radiotherapy or surgical procedures that require highly accurate localization of the target.

### SUMMARY

Certain embodiments of wireless markers containing magnetic transponders are set forth below. It should be understood that these specific embodiments are presented merely to provide the reader with a brief summary of certain forms the invention might take and that these embodiments are not intended to limit the scope of the invention. Indeed, the invention may encompass a variety of embodiments or aspects that may not be set forth below.

In a specific embodiment, the disclosure proposes a change to the materials used in the electrical circuitry of conventional marker transponders. The change significantly increases the radiopacity or radiodensity of transponders and thus improves the visibility of the transponders in radiographic images. The change also improves the electrical performance of transponders.

In a specific embodiment, this disclosure fully or partially replaces copper in the insulated magnet wire of the inductor coil in the electrical circuit of the conventional transponder, with silver. For partial replacement, silver plated copper wire can be used. This replacement enables greater radiopacity and reduced electrical resistance of the coil, which in turn improves the electrical performance of the transponder, an output which enables tracking with a sensing system. Other high-Z, conductive metals, such as gold, platinum, etc., can also be used for full or partial replacement of copper.

Efforts to shrink the overall size of the transponder of markers reduce the amount of dense radiopaque material, i.e. copper, leading to a less radiopaque transponder. In order to image the transponder during treatment or planning using CT or other x-ray imaging systems, this disclosure provides improvement upon the radiopacity of the transponder by substituting copper with a material exhibiting a higher atomic mass, hence increasing radiopacity.

Given enough increase in radiographic visibility and in conjunction with the efforts to shrink the overall size of the transponder, this disclosure allows the transponder to serve as both a smart marker, an electromagnetic transponder for tracking a target with a tracking system, and a more generic radiographic fiducial marker used during CT or other x-ray imaging.

Other aspects and embodiments of the disclosure are described in the section of Detailed Description.

### BRIEF DESCRIPTION OF THE DRAWINGS

These and various other features and advantages will become better understood upon reading of the following detailed description in conjunction with the accompanying drawings and the appended claims provided below, where:

FIG. 1 is an isometric view of a wireless marker in accordance with some embodiments of the disclosure, with a section cut away showing internal components;

FIG. 2 is a cross-sectional view of the marker of FIG. 1 taken along line 2-2;

FIG. 3 is a cross-sectional view of the marker of FIG. 2 taken along line 3-3; and

FIG. 4 is a partial x-ray image showing the visibility of an exemplary marker in accordance with some embodiments of the disclosure.

### DETAILED DESCRIPTION

Various embodiments of wireless markers and methods of imaging using the markers are described. It is to be understood that the disclosure is not limited to the particular embodiments described as such may, of course, vary. An aspect described in conjunction with a particular embodiment is not necessarily limited to that embodiment and can be practiced in any other embodiments.

Embodiments of the disclosure may be described with reference to the figures. It should be noted that some figures are not necessarily drawn to scale. The figures are only intended to facilitate the description of specific embodiments, and are not intended as an exhaustive description or as a limitation on the scope of the disclosure. Further, in the following description, specific details such as examples of specific materials, dimensions, processes, etc. may be set forth in order to provide a thorough understanding of the disclosure. It will be apparent to one of ordinary skill in the art however, that some of these specific details may not be employed to practice embodiments of the disclosure. In other instances, well known components or process steps may not be described in detail in order to avoid unnecessarily obscuring the embodiments of the disclosure. All technical and scientific terms used herein have the meaning as commonly understood by one of ordinary skill in the art unless specifically defined otherwise.

As used herein, the term “wireless marker” refers to an embodiment of the disclosure where the maker is not physically connected via wires to an outside energy source for excitation of the marker or to a tracking system for transmitting signals generated by the marker.

As used herein, the term “patient” refers to a human, animal, or any other object of interest in which a wireless marker of this disclosure can be implanted according to embodiments of the disclosure.

In general, this disclosure provides a wireless marker including a magnetic transponder that improves the visibility of the marker in radiographic images and the electrical performance of the marker for tracking with a tracking system. The magnetic transponder comprises a ferromagnetic core and a coil of a conductive wire around the ferromagnetic core configured to generate a wirelessly transmitted magnetic field in response to wirelessly transmitted excitation energy.

In some embodiment, the material of the conductive wire of the coil is chosen to enhance the visibility of the marker in radiographic imaging. The material of the conductive wire of the coil may further be chosen to improve the electrical performance of the transponder. For example, the conductive wire of the coil may comprise a high-Z material having an atomic number greater than 30. The conductive wire of the coil can be composed or made of a high-Z material. Alternatively, the conductive wire of the coil can be made of a low-Z material plated with a high-Z material. Alternatively, the conductive wire of the coil can be made of an alloy of one or more of low-Z and high-Z metals.

Suitable high-Z materials include and are not limited to silver, gold, and platinum. Therefore, in exemplary embodiments, the conductive wire of the coil can be composed of silver, gold, or platinum. In a preferred embodiment, the conductive wire of the coil is made of silver due to its low electrical resistivity and high radiopacity.

In some exemplary embodiments, the conductive wire of the coil is made of silver, gold, or platinum plated copper. By way of example, the plated copper may include about 95% copper and about 5% silver, gold or platinum. In a preferred embodiment, the conductive wire of the coil is made of silver plated copper comprising about 95% copper and about 5% silver.

The increased radiographic visibility added to the transponder allows the marker to be used as both a more general fiducial marker during CT or other x-ray imaging and a smart marker for tracking a target with a tracking system. Accordingly, in another aspect of the disclosure, a method of localizing a target in a patient is provided. According to the method, a marker is placed at a position relative to a target in a patient. The marker comprises an encapsulation member and a magnetic transponder in the encapsulation member. The magnetic transponder comprises a ferromagnetic core and a coil of a conductive wire around the ferromagnetic core configured to generate a wirelessly transmitted magnetic field signal in response to wirelessly transmitted excitation energy. The conductive wire comprises a high-Z material which enhances the visibility of the transponder in radiographic images. The marker is then imaged using a radiographic imaging system. The imaging can be carried out with a computed tomography (CT) system. The imaging can also be carried out with other x-ray imaging systems using MV or kV x-rays for image acquisition.

The marker may further be excited with excitation energy wirelessly transmitted to the marker. Upon excitation, the marker generates a resonating magnetic field. The resonating magnetic field signal can be wirelessly transmitted and measured with a sensing system external to the patient. The measured signal can then be processed to track the locations of the marker.

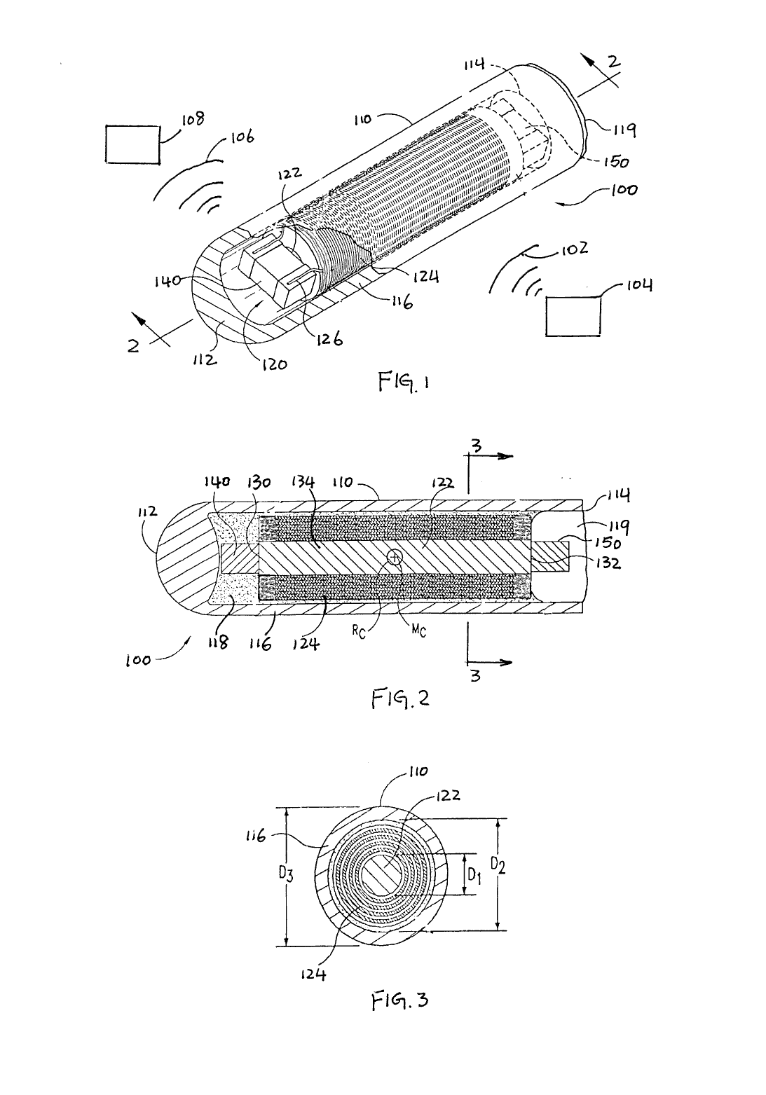

Referring now to FIGS. 1-3, embodiments of wireless markers of the disclosure will now be described. FIG. 1 is an isometric view of an exemplary wireless marker 100 with a section cut away showing internal components. FIG. 2 is a cross-sectional view of the marker 100 of FIG. 1 taken along line 2-2. FIG. 3 is a cross-sectional view of the marker 100 of FIG. 2 taken along line 3-3. In general, the marker 100 is small sized, biologically inert, and can be implanted in a patient or otherwise placed at a position relative to a target in the patient. The marker 100 includes an encapsulation member 110 and a magnetic transponder 120 in the encapsulation member 110. The transponder 120 can be excited by magnetic energy 102 wirelessly transmitted to the marker 100 from a source 104 external to the patient, and generate a magnetic field signal 106 that is wirelessly detectable by a sensing system 108 external to the patient. The marker 100 has enhanced radiodensity and can be detected by an x-ray imaging system (not shown).

The encapsulation member 110 may be strong, rigid, and biocompatible to help protect the magnetic transponder 120 from mechanical damage. For use in medical procedures in which the marker 100 may be permanently implanted in a patient, the encapsulation member 110 encapsulates the magnetic transponder 120 in part to protect the patient's tissues from exposure to any non-biocompatible materials that may be used to optimize the marker signals. The encapsulation member 110 also insulates the transponder 120 from bodily fluids that may cause corrosion or oxidation of the transponder and affect its performance. The encapsulation member 110 may hermetically seal the transponder 120 without adversely affecting the signal element or its emitted signal. The encapsulation member 110 can be made of plastics, ceramics, glass or other suitable biocompatible materials. The encapsulation member 110 may have features such as barbs to anchor the encapsulation member in soft tissue or an adhesive for attaching the encapsulation member externally to the skin of a patient. In some embodiments, the encapsulation member may include radiopaque or radiation opaque material such as leaded glass, high-Z material laced epoxy, Gorilla glass etc. to enhance radiographic visibility of the marker.

In the exemplary embodiment shown in FIGS. 1-3, the encapsulation member 110 may be a biocompatible capsule or vial having a closed end 112, open end 114, and a generally cylindrical wall 116. The transponder 120 may be placed into the vial and an encapsulation material 118, such as a quick-curing adhesive or epoxy, may be injected into the vial to fully encase the transponder 120. Alternatively, a potting material such as an adhesive can be used to secure the transponder in the vial. After the transponder 120 is placed in the vial, the open end 114 of the vial can be sealed by a sealant 119 such as the epoxy or other material. The encapsulation member 110 can also in other configurations and shape. For example, in alternative embodiments, the encapsulation member 110 may include an encapsulation sleeve and the transponder can be placed inside of the sleeve. The transponder can be completely encased in the encapsulation sleeve by a biocompatible material, such as UV-cured or heat-cured epoxy.

The transponder 120 produces a magnetic field signal 106 in response to excitation energy 102. As shown in FIGS. 1-3, the transponder 120 includes a core 122 and a coil 124 of an insulated conductive wire 126 around the core 122. The core 122 is composed of a material having a suitable magnetic permeability. For example, the core 122 may be a ferromagnetic element composed of ferrite or other materials having a relative permeability greater than 1. The core 122 includes a first end 130, a second end 132, and an elongate portion 134 around which the coil 124 of the conductive wire 126 is disposed. In some embodiments, the transponder 120 may include multiple cores to respond selected excitation magnetic fields with different frequencies.

The coil 124 may be made up of a plurality of windings of small diameter, insulated conductive wire 126. For example, the coil 124 may comprise approximately 100-3,000 windings tightly wound around the elongate portion 134 of the core 122. The number of windings in the coil 124 depends upon the wire type, the wire size, the wire shape, the wind geometry, the core size, the core's material, the core's geometry, the inductance required to tune the transponder 120 or the frequency of the magnetic field the transponder 120 responds. The tightly packed coil 124 around the ferromagnetic core 122 allows for a high inductance value to be achieved for the miniature marker assembly's small volume. In an exemplary embodiment, the insulated conductive wire 126 can be hot air or alcohol bonded wire having a gauge of approximately 45-52.

In some embodiments, the transponder 120 may further include a capacitor 140 coupled to the coil 124 at the first end 130 of the core 122. The combination of the capacitor 140 and the plurality of windings of the conductive wire 126 of the coil 124 may resonate at a selected frequency in response to the excitation energy 102. The inclusion of the capacitor 140 in the transponder 120 is optional. In some embodiments, the plurality of windings of the coil 124 can resonate at a selected frequency solely using the parasitic capacitance of the windings without having a capacitor. Therefore, the coil 124 can define a signal transmitter that generates an alternating magnetic field at a selected resonate frequency in response to the excitation energy either by itself or in combination with the capacitor 140.

In some embodiments, the transponder 120 may further include a module 150 at the second end 132 of the core 122 opposite to the capacitor 140 at the first end 130. The module 150 is preferably configured to be symmetrical with respect to the capacitor 140 of the transponder 120. The module 120 may be configured to produce a similar radiographic image as the capacitor 140 in an x-ray. In one embodiment, the module 150 may be configured such that the magnetic centroid (Mc) of the marker 120 is at least substantially coincident with the radiographic centroid (Rc) of the marker 120. In other embodiments that use CT or other types of imaging modalities, the module 150 may be configured to produce a symmetrical image relative to the capacitor 140. For example, the module 150 can be another capacitor identical to the capacitor 140 that may or may not be electrically coupled to the coil 124. In other embodiments, the module 150 can be an electrically inactive element that is not electrically connected to the resonating circuit or another type of electrically active element that is electrically coupled to the resonating circuit. Suitable electrically inactive modules 150 include ceramic blocks shaped like the capacitor 140. In either case, one purpose of the module 150 is to have the same characteristics as the electrically active capacitor 140 in x-ray, CT, and other imaging techniques.

The overall size of the marker 100 may range approximately from 1.0 to 3.0 mm in diameter and from 3.0 to 30 mm in length. By way of example, the core 122 of the ferromagnetic element may be a ferrite rod having a diameter D₁ of approximately 0.20-0.70 mm and a length of approximately 2.0-20 mm. The ferromagnetic element may have other cross-sectional configurations in other embodiments. For example, an extruded ferrite rod can have an elliptical, oval or polygonal cross section. The coil 124 may have an inner diameter of approximately 0.20-0.80 mm and an outer diameter D₂ of approximately 0.6-1.4 mm. The encapsulation member 110 may have an outer diameter D₃ of approximately 1.0-3.0 mm. It should be noted that the specific dimensions are provided herein for a thorough understanding of embodiments of the disclosure. These specific details may not be employed to practice the invention and the scope of the appended claims is not so limited. In other embodiments, the core 122, coil 124, and the encapsulation member 110 may have different dimensions.

There have been efforts to shrink the overall size of transponders and/or markers for implantation in the patient permanently or for extended period of time. However, shrinking the transponder overall size also reduces the amount of dense material, e.g., copper wire 126 of the coil 124, leading to a less radiopaque transponder. As described above, accurately determining the location of a marker relative to a target is a precondition for accurately tracking the target based on the resonating magnetic field generated by the implanted marker. Therefore, it would be desirable to improve the radiopacity of the marker in order to detect the marker using CT or other radiographic imaging system.

Therefore, according to embodiments of the disclosure, the conductive wire 126 of the coil 124 of the transponder 120 may comprise a high-Z material to enhance the visibility of the transponder 120 in radiographic images. By way of example, the high-Z material may have an atomic number greater than 30. Suitable high-Z conductive materials include and are not limited to silver, gold, platinum, tungsten, osmium, and so on.

In some exemplary embodiments, the conductive wire 126 of the coil 124 comprises silver, gold, or platinum. In a particular embodiment, the conductive wire 126 of the coil 124 comprises silver due to its low electrical resistivity and high radiopacity.

The conductive wire 126 of the coil 124 may be composed or made up of a high-Z material. Alternatively, the conductive wire 126 of the coil 124 can be made of a low-Z material plated with a high-Z material. In alternative embodiments, the conductive wire 126 of the coil 124 can be made of an alloy of one or more of low-Z and high-Z metals.

By way of example, the conductive wire 126 of the coil 124 may be made of silver, gold, or platinum. Alternatively, the conductive wire 126 of the coil 124 may be copper plated with silver, gold, or platinum. For example, the conductive wire 126 of the coil 124 may be made of about 80 to 95% by weight of copper and 5 to 20% by weight of silver, gold, or platinum.

As an example, plated copper including about 95% copper and about 5% silver, gold or platinum may be used as the conductive wire 126 of the coil 124. In a particular embodiment, silver plated copper including about 95% copper and about 5% silver is used as the conductive wire 126 of the coil 124.

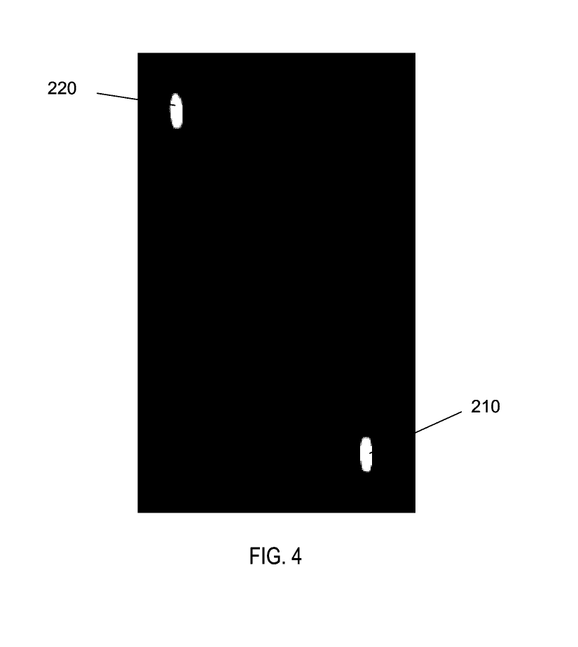

FIG. 4 is an x-ray image showing the visibility of an exemplary marker 210 including a coil of copper wire and an exemplary marker 220 including a coil of silver plated copper wire (95% copper and 5% silver) on the x-ray image. As measured, the maximal Hounsfield Units over the area of the marker increased by approximately 14% when the copper wire was replaced with the silver plated copper wire.

The various embodiments of the wireless markers of this disclosure have increased radiodensity and are highly suitable for use in CT or other x-ray imaging. The improved visibility of the markers in radiographic images allows the markers to be used as generic radiographic fiducial markers. Alternatively or in addition, the markers have improved electrical performance as magnetic transponders for tracking a target with a sensing system.

In use, one or more markers can be implanted in a patient at or near a target using a suitable implantation system. For example, U.S. Pat. No. 8,011,508 B2 describes various embodiments of packaged systems for percutaneous implantation of miniature wireless markers in a patient, the disclosure of which is incorporated herein by reference in its entirety. It should be noted that one or more markers of the disclosure can also be attached to the skin of a patient or secured to an immobilization device on a patient support when in use.

The relative location of the marker with respect to the target in the patient and/or its profile and orientation can be determined using a radiation system such as CT or other x-ray imaging systems. Various x-ray imaging systems are known and therefore their detailed description is omitted here to avoid unnecessary obscuring of the description of this disclosure. Briefly and in general, an x-ray imaging system includes a radiation source, an image acquisition device, and a patient support structure. The radiation source can be a source dedicated for imaging acquisition which typically operates at kilovoltages in producing x-rays. The radiation source may also operate at megavoltages to produce x-rays for either treatment or imaging, or for both. The operation voltage of the radiation source can be adjusted to provide radiation with energy suitable for image acquisition by an image acquisition device. In operation, x-ray radiation from the source irradiates the body portion containing the target and the marker. Radiation is attenuated by the target, the marker, and other features of the body portion as it propagates in the patient. Radiation that passes through the body portion is received by the image acquisition device, which generates image data signals indicative of the structure of the target, the marker, and other features of the body portion. The image data signals may be processed using suitable algorithms known in the art and the processed images may be displayed on a display, which shows the relative location the marker with respect to the target, and the profile and orientation of the marker. The increased radiodensity of the markers of the disclosure allows them to be suitably used with radiation systems producing x-rays having megavoltage and/or kilovoltage energy levels. U.S. Pat. Nos. 5,970,115, 7,291,842, 7,816,651 and 8,552,386 disclose various embodiments of x-ray imaging systems with which the markers of this disclosure can be used. The disclosures of U.S. Pat. Nos. 5,970,115, 7,291,842, 7,816,651 and 8,552,386 are incorporated herein by reference in their entirety.

In tracking a marker, the magnetic transponder of the marker can be excited by magnetic energy. In response to the excitation magnetic energy, the transponder generates a magnetic field signal that is detectable by a sensor array. As shown in FIG. 1, the excitation energy 102 can be transmitted wirelessly to the transponder 120 from a source 104 located externally with respect to the patient. Likewise, the magnetic field signal 106 generated by the transponder 120 can be transmitted wirelessly and detected by a sensor array 108 located externally respect to the patient. Marker tracking systems are known and therefore their detailed description is omitted here to avoid unnecessary obscuring of the description of this disclosure. Briefly and in general, a marker tracking system includes an array of sensors which may be located exterior of the patient's body. The sensors may detect and measure the signal produced by the transponder in response to the excitation energy. The sensors may be positioned in a fixed, selected geometry relative to each other, so the array defines a fixed reference coordinate system from which location and movement are calculated. The sensors may be operatively coupled to a computer controller that receives the measurement information from each sensor and determines the actual location of the markers within the patient's body relative to the sensors. The sensors may be polled multiple times in a specified time period e.g. twelve or more times per minute to track the marker and determine the actual position of the target within the patient's body relative to the sensor array. Accordingly, the actual position of the target can be monitored in real time when the patient is positioned adjacent to the sensor array. U.S. Pat. No. 9,072,895 B2 describes various embodiments of a marker tracking and monitoring system, the disclosure of which is incorporated herein by reference in their entirety.

Embodiments of markers including magnetic transponders and methods of imaging and monitoring using the markers have been described. Those skilled in the art will appreciate that various other modifications may be made within the spirit and scope of the invention. All these or other variations and modifications are contemplated by the inventors and within the scope of the invention.

What is claimed is:

1. A marker comprising:
an encapsulation member; and
a magnetic transponder in the encapsulation member configured to generate a wirelessly transmitted magnetic field in response to wirelessly transmitted excitation energy, wherein the magnetic transponder comprises:
a ferromagnetic core; and
a coil of a conductive wire around the ferromagnetic core, the conductive wire comprising a high-Z material enhancing visibility of the transponder in radiographic images, the high-Z material having an atomic number greater than 30.

2. The marker of claim 1, wherein the conductive wire of the coil is made of the high-Z material.

3. The marker of claim 1, wherein the conductive wire of the coil is made of silver, gold, or platinum.

4. The marker of claim 1, wherein the conductive wire of the coil is made of silver.

5. The marker of claim 1, wherein the conductive wire of the coil comprises copper and the high-Z material.

6. The marker of claim 1, wherein the conductive wire of the coil comprises copper and silver, gold, or platinum.

7. The marker of claim 1, wherein the conductive wire of the coil comprises about 80 to 95% by weight of copper and about 5 to 20% by weight of the high-Z material.

8. The marker of claim 1, wherein the conductive wire of the coil comprises about 80 to 95% by weight of copper and 5 to 20% by weight of silver, gold, or platinum.

9. The marker of claim 1, wherein the conductive wire of the coil comprises about 95% by weight of copper and 5% by weight of silver.

10. The marker of claim 1, wherein the magnetic transponder further comprises a capacitor disposed at a first end of the magnetic transponder electrically coupled to the coil of the conductive wire.

11. The marker of claim 10, wherein the magnetic transponder further comprises a module disposed at a second end of the magnetic transponder, wherein the module has similar radiographic characteristics as the capacitor in a radiographic image.

12. The marker of claim 10, wherein the encapsulation member is biologically inert.

13. The marker of claim 10, wherein the encapsulation member comprises a radiopaque material.

14. The marker of claim 10, wherein the encapsulation member defines a generally cylindrical shape having an outer diameter ranging approximately from 1.0 mm to 3.0 mm and a length ranging approximately from 3.0 mm to 30 mm.

15. A method of locating a target in a patient, comprising:
placing a marker at a position relative to a target, wherein the marker comprises an encapsulation member and a magnetic transponder in the encapsulation member configured to generate a wirelessly transmitted magnetic field in response to wirelessly transmitted excitation energy, the magnetic transponder comprising a ferromagnetic core and a coil of a conductive wire around the ferromagnetic core, the conductive wire of the coil comprising a high-Z material enhancing visibility of the transponder in radiographic images, the high-Z material having an atomic number greater than 30; and
imaging the marker using a radiographic imaging system.

16. The method of claim 15, wherein the imaging is carried out using a computed tomography (CT) system.

17. The method of claim 15, wherein the imaging is carried out using a radiation system producing x-rays having an energy level in megavolts.

18. The method of claim 15, wherein the imaging is carried out using a radiation system producing x-rays having an energy level in kilovolts.

19. The method of claim 15, further comprising
generating magnetic excitation energy that causes the marker to produce a resonating magnetic field signal; and
measuring the resonating magnetic field signal using a sensing system.

20. The method of claim 15, wherein the marker is placed in a patient by percutaneous implantation.

21. The method of claim 15, wherein the conductive wire of the coil comprises silver, gold, or platinum.

22. The method of claim 15, wherein the conductive wire of the coil comprises copper plated with silver, gold, or platinum.

23. The method of claim 15, wherein the conductive wire of the coil is made of silver plated copper.
